(19) United States
(12) Patent Application Publication
(10) Pub. No.: US 2024/0203604 A1
(43) Pub. Date: Jun. 20, 2024

(54) ESTIMATING EFFECTS WITH LATENT REPRESENTATIONS

(71) Applicant: Adobe Inc., San Jose, CA (US)

(72) Inventors: Kunwar Singh, Jalandhar (IN); Vidit Bhatia, Modinagar (IN)

(73) Assignee: Adobe Inc., San Jose, CA (US)

(21) Appl. No.: 18/067,925

(22) Filed: Dec. 19, 2022

(51) Int. Cl. G16H 70/40

(52) U.S. Cl. CPC G16H 70/40

(57) ABSTRACT

In implementations of systems for estimating effects with latent representations, a computing device implements an estimation system to receive input data via a network describing interactions of client devices included in a group of client devices. The estimation system generates a first latent vector representation of a first segment of the client devices and a second latent vector representation of a second segment of the client devices using an encoder of a machine learning model. A change vector is computed based on a difference between the first latent vector representation and the second latent vector representation in a latent space of the machine learning model. The estimation system generates an indication of an effect of a treatment on a third segment of the client devices based on the change vector using a decoder of the machine learning model.

### BACKGROUND

A treatment refers to a controlled change or intervention, and a treatment effect is a causal effect of the treatment on particular outcome. For example, in a clinical trial for a new drug, administration of the new drug to a participant in the clinical trial is the treatment. The treatment effect is a change in the participant's health (if any) as a result of receiving the new drug (e.g., a difference between the participant's health before and after receiving the new drug).

### SUMMARY

Techniques and systems for estimating effects with latent representations are described. In an example, a computing device implements an estimation system to receive input data via a network describing interactions of client devices included in a group of client devices. In this example, the input data includes categorical data and numerical data describing the interactions of the client devices. The estimation system generates a first latent vector representation of a first segment of the client devices and a second latent vector representation of a second segment of the client devices using an encoder of a machine learning model. In some examples, the machine learning model includes a variational autoencoder.

The estimation system computes a change vector based on a difference between the first latent vector representation and the second latent vector representation in a latent space of the machine learning model. An indication is generated of an effect of a treatment based on a third latent vector representation of a third segment of the client devices and the change vector using a decoder of the machine learning model. For example, the estimation system combines the third latent vector representation and the change vector in the latent space and decodes this latent combination using the decoder in order to generate the indication of the effect of the treatment.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures. Entities represented in the figures are indicative of one or more entities and thus reference is made interchangeably to single or plural forms of the entities in the discussion.

FIG. 1 is an illustration of an environment in an example implementation that is operable to employ digital systems and techniques for estimating effects with latent representations as described herein.

FIG. 2 depicts a system in an example implementation showing operation of an estimation module for estimating effects with latent representations.

FIG. 3 illustrates a representation of generating concatenated data.

FIG. 4 illustrates a representation of a machine learning model.

FIG. 5 illustrates a representation of computing a change vector based on latent representations.

FIG. 6 illustrates a representation of generating an indication of an effect of a treatment based on a change vector.

FIG. 7 is a flow diagram depicting a procedure in an example implementation in which an indication of an effect of a treatment is generated based on a change vector.

FIG. 8 is a flow diagram depicting a procedure in an example implementation in which an indication of an effect of a treatment is generated based on a difference between a first latent vector representation and a second latent vector representation.

FIG. 9 illustrates an example system that includes an example computing device that is representative of one or more computing systems and/or devices for implementing the various techniques described herein.

### DETAILED DESCRIPTION

### Overview

A treatment is a controlled change or intervention, and an effect of the treatment is any outcome which is influenced or caused by the controlled change or intervention. It is possible to estimate an effect of a treatment, for example, as an average difference between outcomes before and after the treatment. However, conventional systems for estimating effects of a treatment require information about the treatment (e.g., as a prior) in order to learn to accurately predict effects of the treatment. As a result, conventional systems are limited to estimating effects for a set of known (pre-defined) treatments. In order to overcome this limitation, techniques and systems for estimating effects with latent representations are described.

In an example, a computing device implements an estimation system to receive input data via a network describing interactions of client devices included in a group of client devices. For example, the interactions of the client devices are interactions with digital templates included in a template database, interactions as part of a collaborative digital content editing session, interactions with digital images included in an image database, and so forth. The estimation system uses the input data to train a machine learning model to generate latent vector representations of segments of client devices included in the group of client devices.

In some examples, the machine learning model includes a variational autoencoder, and the estimation system receives the input data as including categorical data describing the interactions of the client devices as well as numerical data describing the interactions of the client devices. In these examples, the estimation system embeds the categorical data using embedding layers of the machine learning model, and then batch normalizes the embedded categorical data. The estimation system concatenates the batch normalized categorical data and the numerical data as a concatenated vector.

For example, the estimation system processes the concatenated vector using an encoder of the machine learning model to generate a first latent vector representation of a first segment of the client devices in a latent space of the model. In this example, the estimation system regularizes the latent space using a Kullback-Leibler divergence loss. As part of training the machine learning model on the input data to generate the latent vector representations in the latent space, the estimation system uses a binary cross-entropy loss for the categorical data and a mean squared loss for the numerical data. For instance, the estimation system trains the machine learning model on the input data without an indication of a treatment which has been received by the first segment of the client devices.

The treatment is a recommendation of a font to use with a digital template included in the template database, an updated version of an application used to collaboratively edit digital content, a recommendation of a digital template to receive a digital image included in the image database, etc. In some examples, the estimation system has information regarding the treatment received by the first segment of the client devices. Notably, in other examples, the estimation system has no information regarding the treatment.

The estimation system implements the encoder of the machine learning model to generate a second latent vector representation of a second segment of the client devices in the latent space. In one example, the second segment of the client devices has not received the treatment. The estimation system computes a change vector based on an average difference between the first latent vector representation and the second latent vector representation in the latent space of the machine learning model. Since the first segment of the client devices received the treatment and the second segment of the client devices did not receive the treatment, the change vector is representative of an effect of the treatment in the latent space.

For example, the estimation system is capable of leveraging the change vector and the machine learning model to generate a transaction vector for a third segment of the client devices that has not received the treatment. To do so in one example, the estimation system generates a third latent vector representation of the third segment of the client devices using the encoder of the machine learning model. The third latent vector representation is combined with the change vector in the latent space, and the estimation system generates the transaction vector by decoding this combined latent representation using a decoder of the machine learning model.

The transaction vector approximates the third segment after receiving the treatment, and the estimation system is capable of generating the transaction vector without having any information about the treatment. This is not possible using conventional systems that require information about the treatment in order to accurately estimate an effect of the treatment. In addition to leveraging the change vector to estimate effects of treatments, the estimation system is also capable of using the change vector to implement other functionality (e.g., auto-segmentation, segment discovery, segment expansion, etc.) which is a further improvement relative to the conventional systems.

In the following discussion, an example environment is first described that employs examples of techniques described herein. Example procedures are also described which are performable in the example environment and other environments. Consequently, performance of the example procedures is not limited to the example environment and the example environment is not limited to performance of the example procedures.

### Example Environment

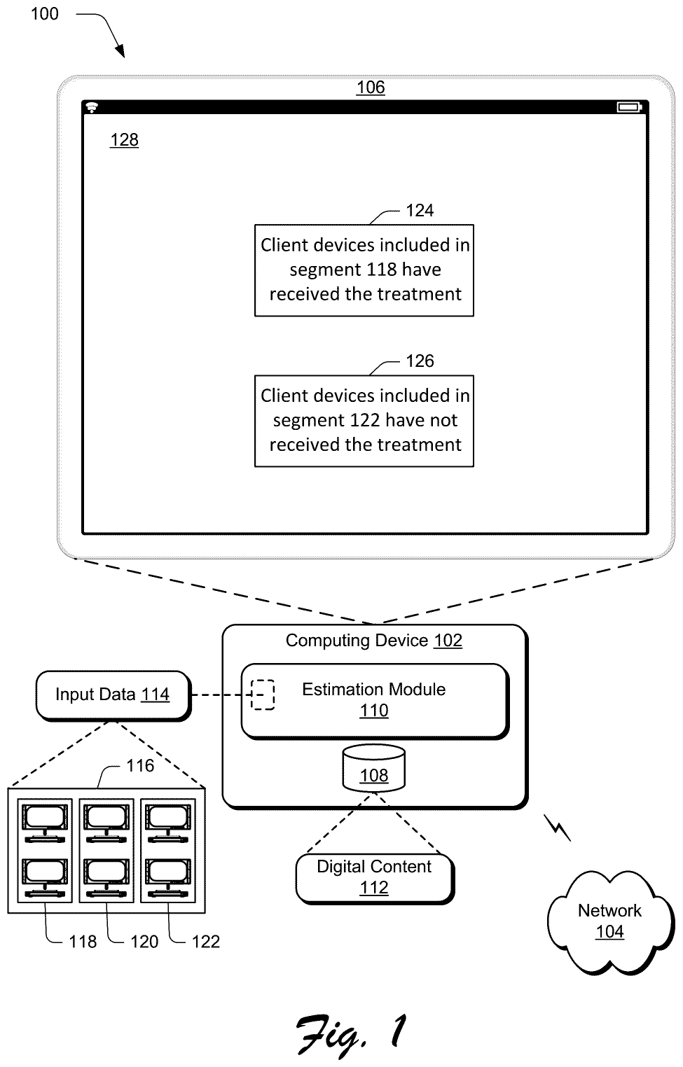

FIG. 1 is an illustration of an environment 100 in an example implementation that is operable to employ digital systems and techniques as described herein. The illustrated environment 100 includes a computing device 102 connected to a network 104. The computing device 102 is configurable as a desktop computer, a laptop computer, a mobile device (e.g., assuming a handheld configuration such as a tablet or mobile phone), and so forth. Thus, the computing device 102 is capable of ranging from a full resource device with substantial memory and processor resources (e.g., personal computers, game consoles) to a low-resource device with limited memory and/or processing resources (e.g., mobile devices). In some examples, the computing device 102 is representative of a plurality of different devices such as multiple servers utilized to perform operations “over the cloud.”

The illustrated environment 100 also includes a display device 106 that is communicatively coupled to the computing device 102 via a wired or a wireless connection. A variety of device configurations are usable to implement the computing device 102 and/or the display device 106. The computing device 102 includes a storage device 108 and an estimation module 110. For instance, the storage device 108 is illustrated to include digital content 112 such as digital images, electronic documents, digital videos, etc.

The estimation module 110 is illustrated as having, receiving, and/or transmitting input data 114. In an example, the estimation module 110 receives the input data 114 via the network 104. As shown, the input data 114 describes interactions of client devices included in a group 116 of client devices. In this example, the group 116 of client devices includes a first segment 118 of the client devices, a second segment 120 of the client devices, and a third segment 122 of the client devices.

Consider a first example in which the interactions of the client devices included in the group 116 are interactions with a font service via the network 104. For instance, the font service includes a database of thousands of different fonts. In the first example, users of the client devices manipulate an input device (e.g., a mouse, a keyboard, a stylus, a touchscreen, etc.) to interact with the font service such as to identify fonts included in the database having particular visual features, to create new fonts to be included in the database, to identify fonts included in the database that are visually similar to a particular font that is not included in the database, and so forth.

Consider a second example in which the interactions of the client devices included in the group 116 are interactions as part of a collaborative digital content editing session conducted via the network 104. In this second example, users of the client devices manipulate the input device to participate in the collaborative digital content editing session such as by adding objects to digital content, changing visual features of objects included in the digital content, commenting in relation to an editing operation performed on the digital content by another participant in the collaborative editing session, etc. The computing device 102 implements the estimation module 110 to estimate an effect of a treatment on the group 116 of client devices. For example, the treatment is a controlled change or intervention and the effect of the treatment is any outcome which is influenced or caused by the controlled change or intervention. In some examples, the effect of the treatment on the group 116 of client devices is an average difference between any variable for client devices included in the group 116 that receive the treatment and client devices included in the group 116 that do not receive the treatment.

In the first example, the treatment is a particular recommended font. For example, the estimation module 110 recommends the particular font to the first segment 118 of the client devices and the estimation module 110 does not recommend the particular font to the second segment 120 of the client devices. In this example, the effect of the treatment is use of the particular font to render glyphs of text in a digital template.

In the second example, the treatment is a new version of a web-based digital content editing application that is still being tested before release. Continuing the second example, the estimation module 110 replaces a legacy version of the web-based digital content editing application with the new version for the first segment 118 of the client devices and the estimation module 110 does not replace the legacy version for the second segment 120 of the client devices. For example, the effect of the treatment is a difference between a number of error log entries for the first segment 118 when using the new version of the application and a number of error log entries for the second segment 120 using the legacy version of the application.

In order to estimate the effect of the treatment on the group 116 of client devices, the estimation module 110 leverages a machine learning model. As used herein, the term “machine learning model” refers to a computer representation that is tunable (e.g., trainable) based on inputs to approximate unknown functions. By way of example, the term “machine learning model” includes a model that utilizes algorithms to learn from, and make predictions on, known data by analyzing the known data to learn to generate outputs that reflect patterns and attributes of the known data. According to various implementations, such a machine learning model uses supervised learning, semi-supervised learning, unsupervised learning, reinforcement learning, and/or transfer learning. For example, the machine learning model is capable of including, but is not limited to, clustering, decision trees, support vector machines, linear regression, logistic regression, Bayesian networks, random forest learning, dimensionality reduction algorithms, boosting algorithms, artificial neural networks (e.g., fully-connected neural networks, deep convolutional neural networks, or recurrent neural networks), deep learning, etc. By way of example, a machine learning model makes high-level abstractions in data by generating data-driven predictions or decisions from the known input data.

In an example, the machine learning model includes a variational autoencoder and the estimation module 110 receives and processes the input data 114 to generate a first latent vector representation of the first segment 118 of the client devices and a second latent vector representation of the second segment 120 of the client devices in a latent space of the variational autoencoder. For example, the estimation module 110 generates the first and second latent vector representations using an encoder of the variational autoencoder. The estimation module 110 computes a change vector based on an average difference between the first latent vector representation and the second latent vector representation in the latent space of the variational autoencoder.

Since the first segment 118 of the client devices received the treatment and because the second segment 120 of the client devices did not receive the treatment, the change vector is representative of an effect of the treatment in the latent space of the variational autoencoder. For example, the estimation module 110 is capable of leveraging the change vector as a representation of the effect of the treatment regardless of whether the treatment is a recommended font as in the first example or whether the treatment is a new version of the web-based digital content editing application as in the second example. Consider an example in which the third segment 122 of the client devices did not receive the treatment and the estimation module 110 is capable of estimating an effect of the treatment on the third segment 122 using the change vector and the variational autoencoder.

To do so, the estimation module 110 generates a third latent vector representation of the third segment 122 of the client devices using the encoder of the variational autoencoder. The estimation module 110 then combines the third latent vector representation of the third segment and the change vector in the latent space of the variational autoencoder. For example, the estimation module 110 decodes the combination in the latent space using a decoder of the variational autoencoder to generate an estimated effect of the treatment for the third segment 122 of the client devices.

For instance, the estimation module 110 is capable of leveraging the change vector and the variational autoencoder to facilitate functionality such as generating indications 124, 126 which are displayed in a user interface 128 of the display device 106. As shown, indication 124 states “Client devices included in segment 118 have received the treatment” and indication 126 states “Client devices included in segment 122 have not received the treatment.” Notably, the estimation module 110 is capable of accurately generating the indications 124, 126 without an indication of the treatment (e.g., without knowledge of the treatment). This is not possible using conventional systems for estimating effects that require knowledge of the treatment as a prior in order to accurately estimate effects.

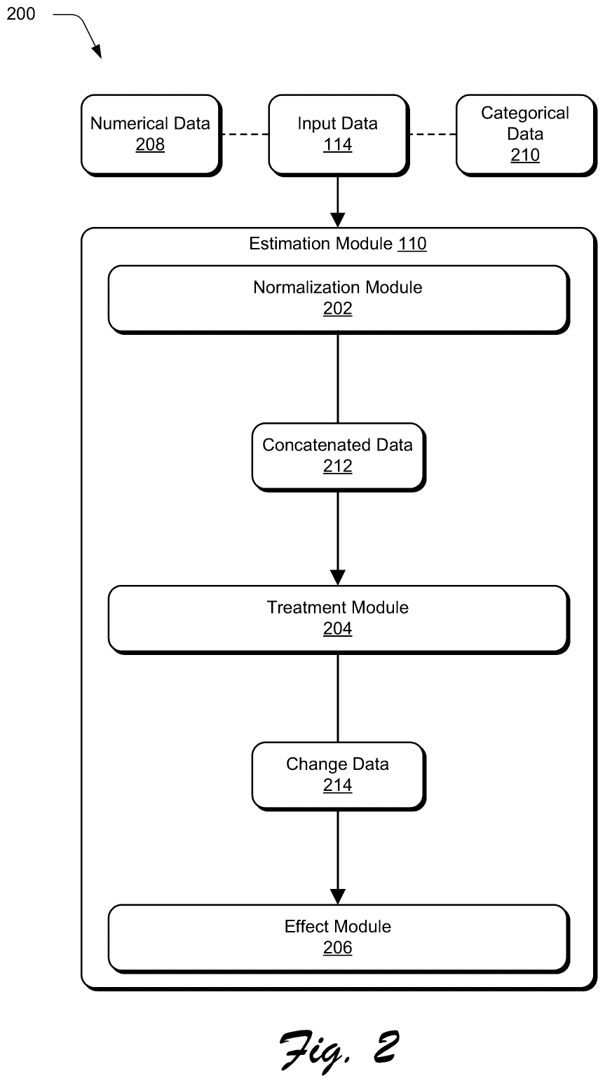

FIG. 2 depicts a system 200 in an example implementation showing operation of an estimation module 110. The estimation module 110 is illustrated to include a normalization module 202, a treatment module 204, and an effect module 206. The estimation module 110 receives the input data 114 describing interactions of the client devices included in the group 116 of client devices which includes numerical data 208 and categorical data 210. For example, the normalization module 202 receives and processes the input data 114 including the numerical data 208 and the categorical data 210 to generate concatenated data 212.

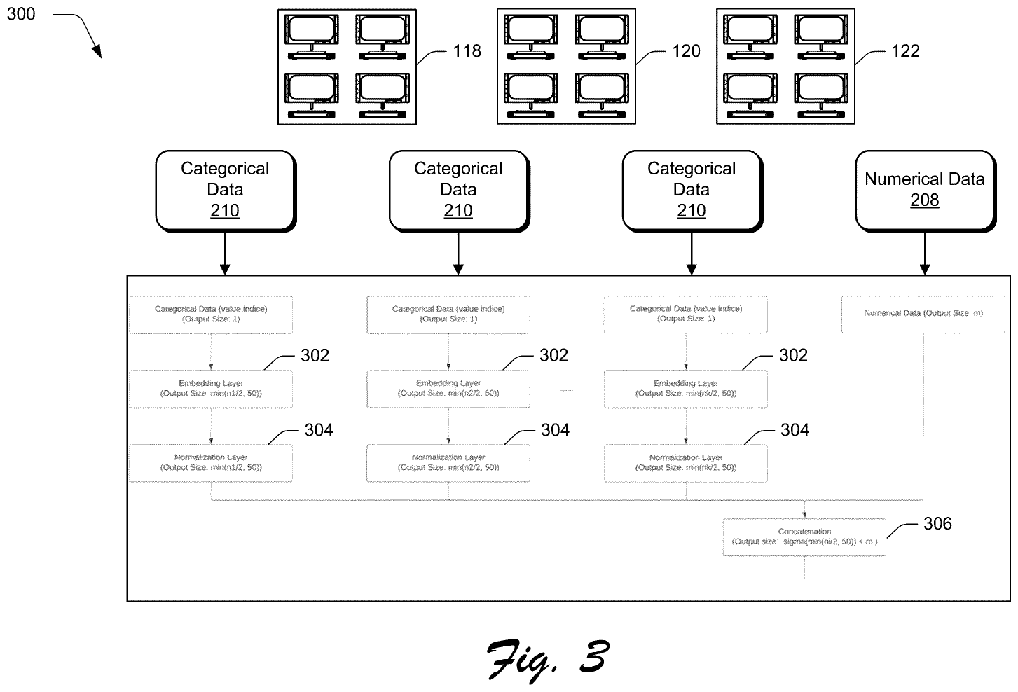

FIG. 3 illustrates a representation 300 of generating concatenated data. As shown, the representation 300 includes the first segment 118 of the client devices, the second segment 120 of the client devices, and the third segment 122 of the client devices. In order to process the numerical data 208 and the categorical data 210 using the machine learning model that includes the variational autoencoder, the normalization module 202 pre-processes the categorical data 210 using embedding layers 302 and a normalization layer 304 (e.g., embedding layers 302 embed the categorical data 210 and the normalization layer 304 batch normalizes the embedded categorical data 210). For example, the normalization module 202 concatenates the batch normalized categorical data 210 with the numerical data 208 (e.g., the numerical data 208 is normalized as received or is already normalized) to generate a concatenated vector 306. In this example, the normalization module 202 generates the concatenated data 212 as describing the concatenated vector 306. The treatment module 204 receives and processes the concatenated data 212 to generate change data 214.

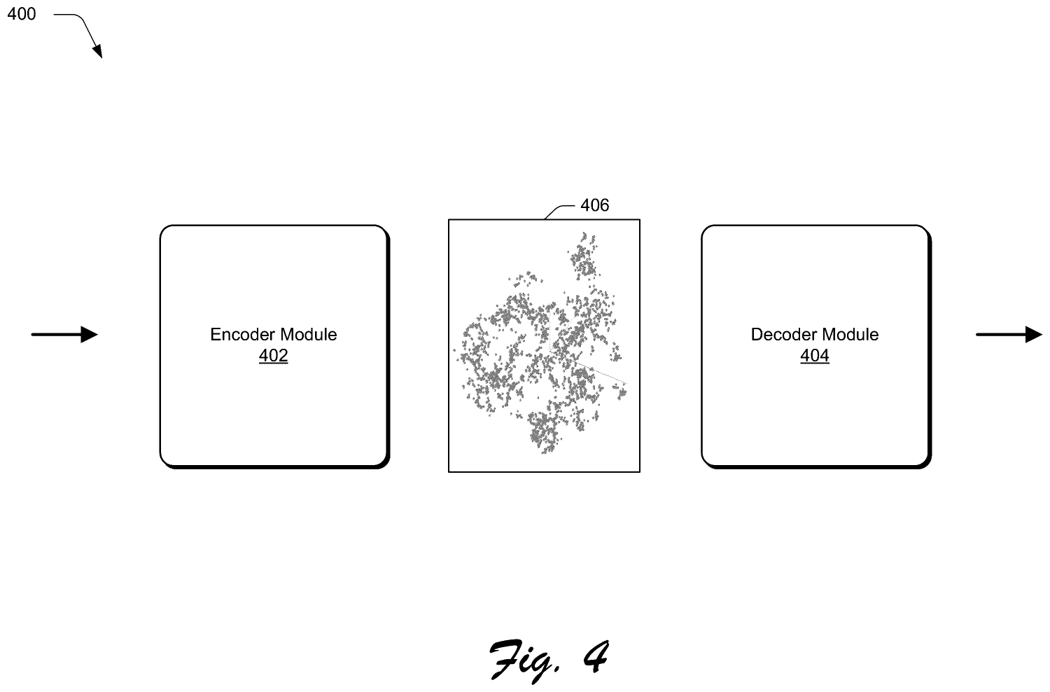

FIG. 4 illustrates a representation 400 of a machine learning model. As shown in the representation 400, the machine learning model includes a variational autoencoder that is illustrated as an encoder module 402, a decoder module 404, and a latent space 406. For example, the estimation module 110 includes the variational autoencoder, and the estimation module 110 processes the concatenated vector 306 described by the concatenated data 212 using an encoder of the variational autoencoder included in the encoder module 402. In this example, the encoder includes a dense layer (e.g., output size 100) with rectified linear unit (ReLU) activation.

A decoder of the variational autoencoder is included in the decoder module 404. For example, the decoder includes dense layers (e.g., output size 100 with ReLU activation) to decode the latent space 406 and an extra dense layer to separately decode the numerical data 208 and the categorical data 210. In this example, the numerical data 208 has linear activation and the categorical data 210 is decoded as its one-hot encoded representation with softmax activation. The estimation module 110 trains the variational autoencoder on the input data 114 without an indication of the treatment. As part of the training, a binary cross-entropy loss is used for the categorical data 210, a mean squared loss is used for the numerical data 208, and a Kullback-Leibler divergence loss is used for regularization of the latent space 406.

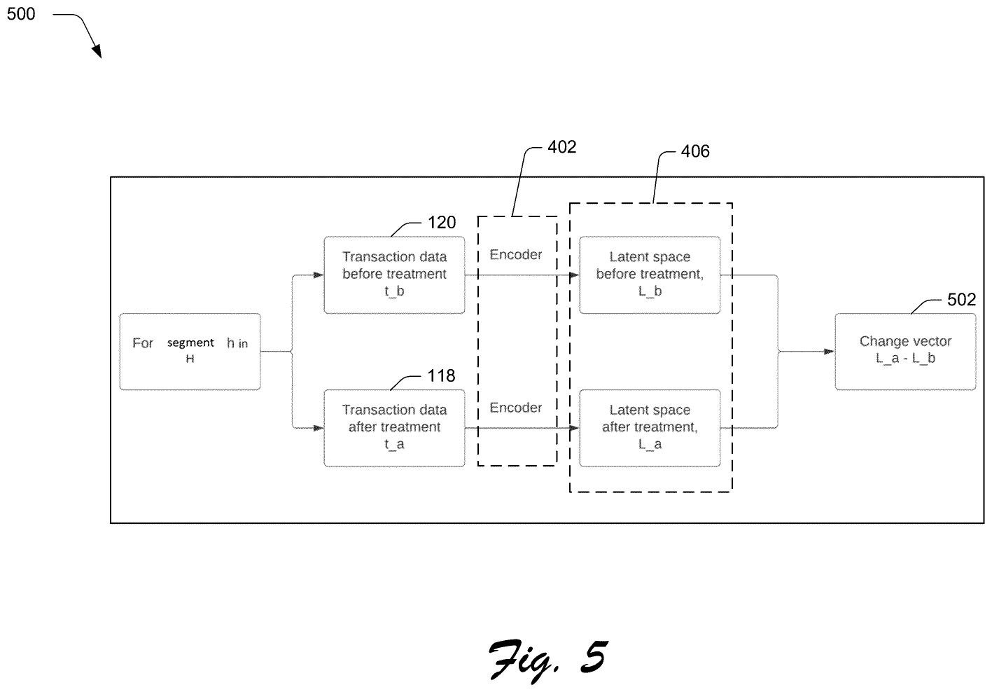

FIG. 5 illustrates a representation 500 of computing a change vector based on latent representations. The treatment module 204 processes the concatenated data 212 using the encoder of the variational autoencoder included in the encoder module 402 to generate a first latent vector representation of the first segment 118 of the client devices in the latent space 406 of the variational autoencoder. For example, the first segment 118 of the client devices has received the treatment and first latent vector representation includes a representation of the treatment in the latent space 406. In this example, the treatment module 204 also uses the encoder included in the encoder module 402 to generate a second latent vector representation of the second segment 120 of the client devices in the latent space 406 of the variational autoencoder. For instance, the second segment 120 of the client devices has not received the treatment and the second latent vector representation does not include a representation of the treatment in the latent space 406.

The treatment module 204 computes a change vector 502 based on a difference between the first latent vector representation and the second latent vector representation in the latent space 406 of the variational autoencoder. In an example, the treatment module 204 computes the change vector 502 as a mean or average difference between the first latent vector representation of the first segment 118 and the second latent vector representation of the second segment 120. Since the treatment has been applied to the first segment 118 and the treatment has not been applied to the second segment 120, the change vector 502 is representative of an effect of the treatment in the latent space 406. For example, the treatment module 204 generates the change data 214 as describing the change vector 502.

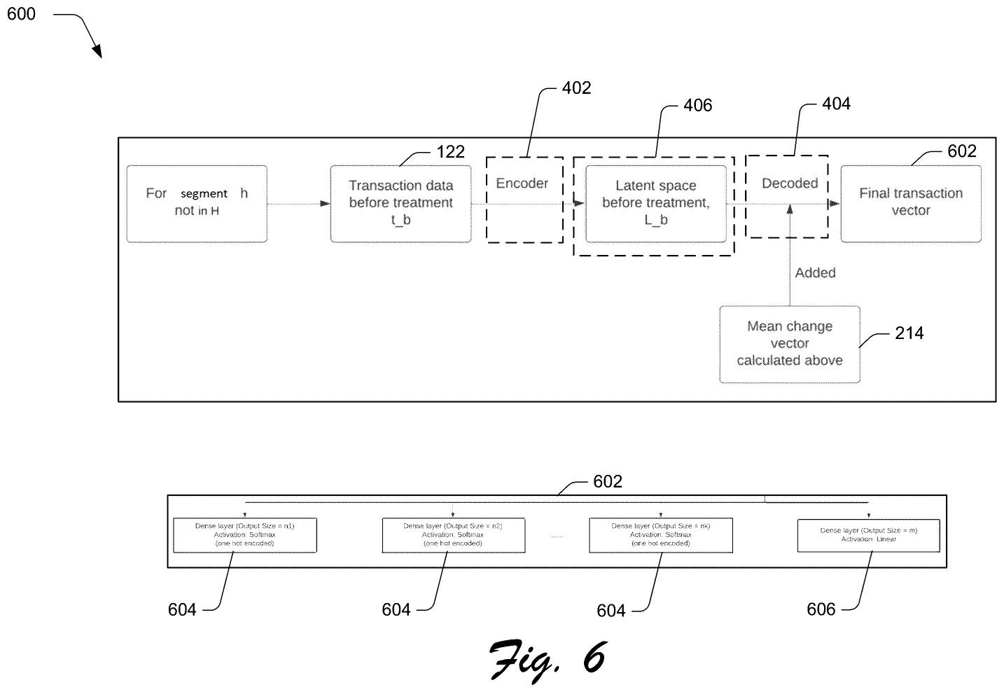

The effect module 206 receives and processes the change data 214 and then leverages the change vector 502 and the variational autoencoder to facilitate a variety of functionality such as estimating an effect of the treatment on the third segment 122 of the client devices. FIG. 6 illustrates a representation 600 of generating an indication of an effect of a treatment based on a change vector. For example, the effect module 206 implements the encoder module 402 to generate a third latent vector representation of the third segment 122 of the client devices in the latent space 406 of the variational autoencoder. In this example, the third segment 122 of the client devices has not received the treatment and the effect module 206 estimates an effect of the treatment on the third segment 122 by combining the third latent vector representation with the change vector 502 described by the change data 214 in the latent space 406.

The effect module 206 uses the decoder of the variational autoencoder included in the decoder module 404 to decode the combination of the third latent vector representation and the change vector 502 in order to generate a final transaction vector 602. As described above, the extra dense layer of the decoder is implemented to separately decode the categorical data 210 as categorical outputs 604 and the numerical data 208 as numerical outputs 606. For example, the effect module 206 generates an indication of the effect of the treatment on the third segment 122 (e.g., the final transaction vector 602) for display in the user interface 128 of the display device 106.

By computing the change vector 502 in the regularized latent space 406 of the variational autoencoder in this manner, the estimation module 110 is capable of estimating effects of treatments on the client devices included in the group 116 of client devices without any knowledge of these treatments. For instance, the estimation module 110 is capable of computing the change vector 502 and generating the final transaction vector 602 to estimate the effect of the treatment on the third segment 122 without any information as to whether the first segment 118 or the second segment 120 received the treatment. In some examples, the estimation module 110 is capable of estimating effects of treatments based on representations in the latent space 406 even if these representations are not directly meaningful outside of the latent space 406. This is not possible in conventional systems that require information about a treatment as a prior in order to accurately estimate effects of the treatment. Additionally, it is to be appreciated that the described systems for estimating effects with latent representations are leverageable to support a variety of additional functionality such as automatic segmentation of the group 116 of client devices, segment discovery, segment expansion, and so forth. For example, by computing differences between latent vector representations in the latent space 406, it is possible for the computing device 102 to implement the estimation module 110 to perform AB testing causal analysis, next action predictions, etc.

In general, functionality, features, and concepts described in relation to the examples above and below are employed in the context of the example procedures described in this section. Further, functionality, features, and concepts described in relation to different figures and examples in this document are interchangeable among one another and are not limited to implementation in the context of a particular figure or procedure. Moreover, blocks associated with different representative procedures and corresponding figures herein are applicable individually, together, and/or combined in different ways. Thus, individual functionality, features, and concepts described in relation to different example environments, devices, components, figures, and procedures herein are usable in any suitable combinations and are not limited to the particular combinations represented by the enumerated examples in this description.

### Example Procedures

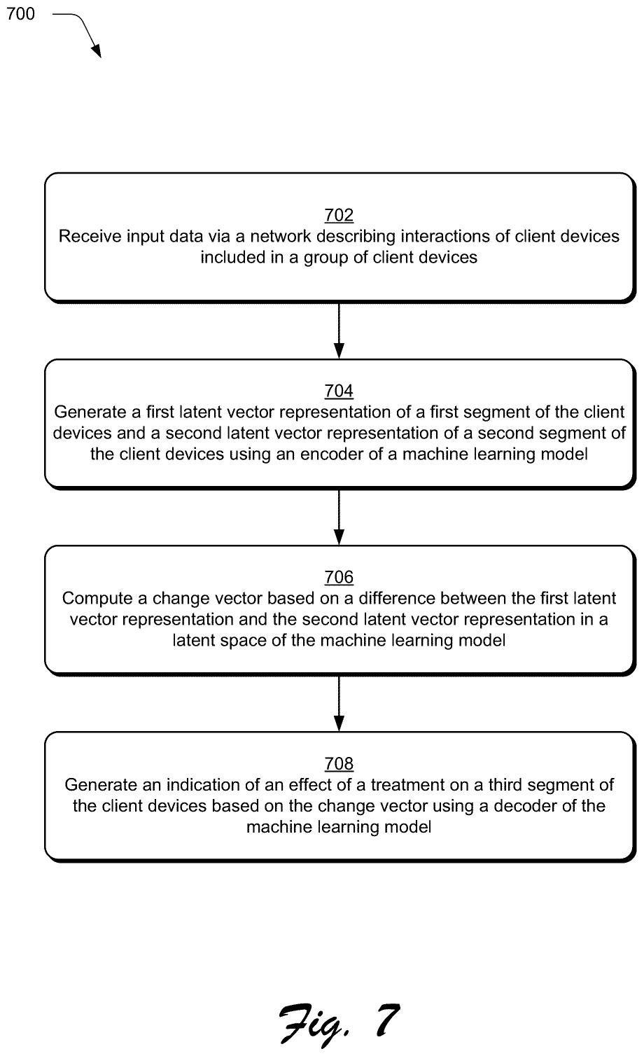

The following discussion describes techniques which are implementable utilizing the previously described systems and devices. Aspects of each of the procedures are implementable in hardware, firmware, software, or a combination thereof. The procedures are shown as a set of blocks that specify operations performed by one or more devices and are not necessarily limited to the orders shown for performing the operations by the respective blocks. In portions of the following discussion, reference is made to FIGS. 1-6. FIG. 7 is a flow diagram depicting a procedure 700 in an example implementation in which an indication of an effect of a treatment is generated based on a change vector.

Input data is received via a network describing interactions of client devices included in a group of client devices (block 702). In some examples, the computing device 102 implements the estimation module 110 to receive the input data. A first latent vector representation of a first segment of the client devices and a second latent vector representation of a second segment of the client devices are generated using an encoder of a machine learning model (block 704). For example, the estimation module 110 generates the first latent vector representation and the second latent vector representation.

A change vector is computed based on a difference between the first latent vector representation and the second latent vector representation in a latent space of the machine learning model (block 706). In an example, the computing device 102 implements the estimation module 110 to compute the change vector. An indication of an effect of a treatment on a third segment of the client devices is generated based on the change vector using a decoder of the machine learning model (block 708). In one example, the estimation module 110 generates the indication of the effect of the treatment.

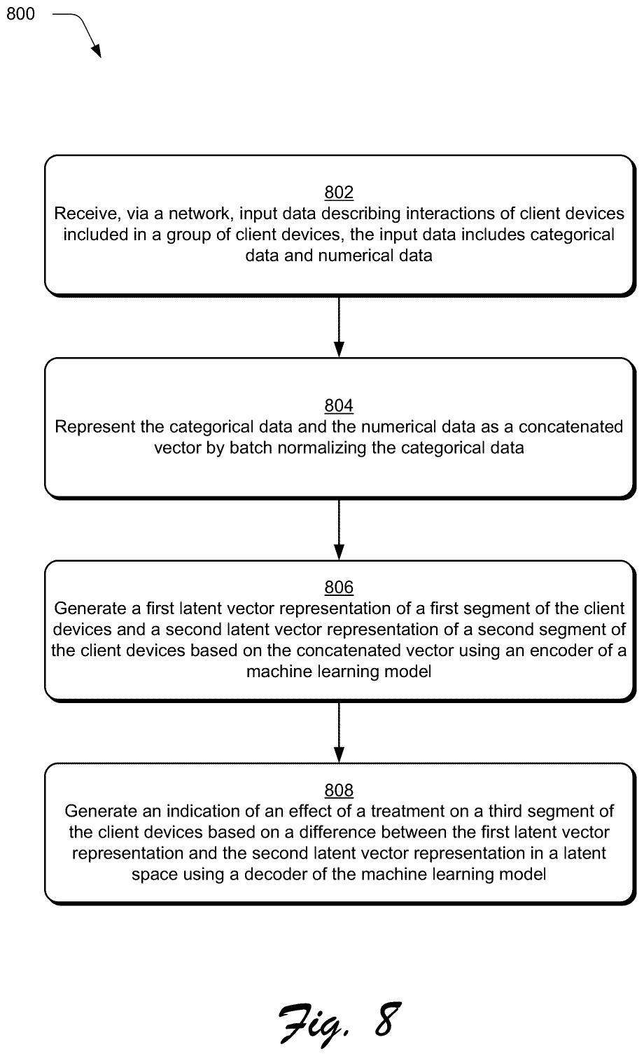

FIG. 8 is a flow diagram depicting a procedure 800 in an example implementation in which an indication of an effect of a treatment is generated based on a difference between a first latent vector representation and a second latent vector representation. Input data is received via a network describing interactions of client devices included in a group of client devices, the input data includes categorical data and numerical data (block 802). For example, the computing device 102 implements the estimation module 110 to receive the input data.

The categorical data and the numerical data are represented as a concatenated vector by batch normalizing the categorical data (block 804). In one example, the estimation module 110 represents the categorical data and the numerical data as the concatenated vector. A first latent vector representation of a first segment of the client devices and a second latent vector representation of a second segment of the client devices are generated based on the concatenated vector using an encoder of a machine learning model (block 806). In some examples, the estimation module 110 generates the first latent vector representation and the second latent vector representation. An indication of an effect of a treatment on a third segment of the client devices is generated based on a difference between the first latent vector representation and the second latent vector representation in a latent space using a decoder of the machine learning model (block 808). In an example, the computing device 102 implements the estimation module 110 to generate the indication of the effect of the treatment.

### Example System and Device

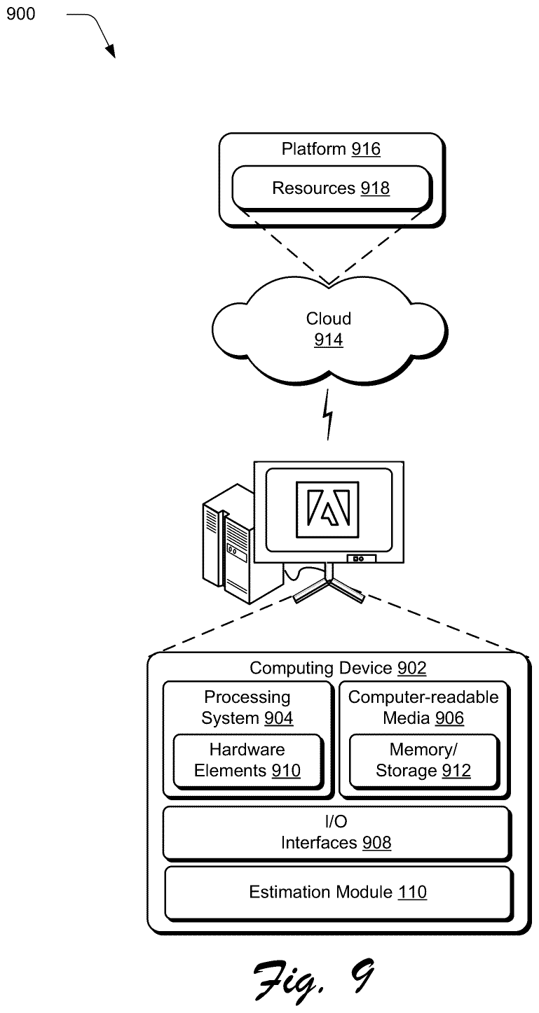

FIG. 9 illustrates an example system 900 that includes an example computing device that is representative of one or more computing systems and/or devices that are usable to implement the various techniques described herein. This is illustrated through inclusion of the estimation module 110. The computing device 902 includes, for example, a server of a service provider, a device associated with a client (e.g., a client device), an on-chip system, and/or any other suitable computing device or computing system.

The example computing device 902 as illustrated includes a processing system 904, one or more computer-readable media 906, and one or more I/O interfaces 908 that are communicatively coupled, one to another. Although not shown, the computing device 902 further includes a system bus or other data and command transfer system that couples the various components, one to another. For example, a system bus includes any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 904 is representative of functionality to perform one or more operations using hardware. Accordingly, the processing system 904 is illustrated as including hardware elements 910 that are configured as processors, functional blocks, and so forth. This includes example implementations in hardware as an application specific integrated circuit or other logic device formed using one or more semiconductors. The hardware elements 910 are not limited by the materials from which they are formed or the processing mechanisms employed therein. For example, processors are comprised of semiconductor(s) and/or transistors (e.g., electronic integrated circuits (ICs)). In such a context, processor-executable instructions are, for example, electronically-executable instructions.

The computer-readable media 906 is illustrated as including memory/storage 912. The memory/storage 912 represents memory/storage capacity associated with one or more computer-readable media. In one example, the memory/storage 912 includes volatile media (such as random access memory (RAM)) and/or nonvolatile media (such as read only memory (ROM), Flash memory, optical disks, magnetic disks, and so forth). In another example, the memory/storage 912 includes fixed media (e.g., RAM, ROM, a fixed hard drive, and so on) as well as removable media (e.g., Flash memory, a removable hard drive, an optical disc, and so forth). The computer-readable media 906 is configurable in a variety of other ways as further described below.

Input/output interface(s) 908 are representative of functionality to allow a user to enter commands and information to computing device 902, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which employs visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 902 is configurable in a variety of ways as further described below to support user interaction.

Various techniques are described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques are implementable on a variety of commercial computing platforms having a variety of processors.

Implementations of the described modules and techniques are storable on or transmitted across some form of computer-readable media. For example, the computer-readable media includes a variety of media that is accessible to the computing device 902. By way of example, and not limitation, computer-readable media includes “computer-readable storage media” and “computer-readable signal media.”

“Computer-readable storage media” refers to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which are accessible to a computer.

“Computer-readable signal media” refers to a signal-bearing medium that is configured to transmit instructions to the hardware of the computing device 902, such as via a network. Signal media typically embodies computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 910 and computer-readable media 906 are representative of modules, programmable device logic and/or fixed device logic implemented in a hardware form that is employable in some embodiments to implement at least some aspects of the techniques described herein, such as to perform one or more instructions. Hardware includes components of an integrated circuit or on-chip system, an application-specific integrated circuit (ASIC), a field-programmable gate array (FPGA), a complex programmable logic device (CPLD), and other implementations in silicon or other hardware. In this context, hardware operates as a processing device that performs program tasks defined by instructions and/or logic embodied by the hardware as well as a hardware utilized to store instructions for execution, e.g., the computer-readable storage media described previously.

Combinations of the foregoing are also employable to implement various techniques described herein. Accordingly, software, hardware, or executable modules are implementable as one or more instructions and/or logic embodied on some form of computer-readable storage media and/or by one or more hardware elements 910. For example, the computing device 902 is configured to implement particular instructions and/or functions corresponding to the software and/or hardware modules. Accordingly, implementation of a module that is executable by the computing device 902 as software is achieved at least partially in hardware, e.g., through use of computer-readable storage media and/or hardware elements 910 of the processing system 904. The instructions and/or functions are executable/operable by one or more articles of manufacture (for example, one or more computing devices 902 and/or processing systems 904) to implement techniques, modules, and examples described herein.

The techniques described herein are supportable by various configurations of the computing device 902 and are not limited to the specific examples of the techniques described herein. This functionality is also implementable entirely or partially through use of a distributed system, such as over a “cloud” 914 as described below.

The cloud 914 includes and/or is representative of a platform 916 for resources 918. The platform 916 abstracts underlying functionality of hardware (e.g., servers) and software resources of the cloud 914. For example, the resources 918 include applications and/or data that are utilized while computer processing is executed on servers that are remote from the computing device 902. In some examples, the resources 918 also include services provided over the Internet and/or through a subscriber network, such as a cellular or Wi-Fi network.

The platform 916 abstracts the resources 918 and functions to connect the computing device 902 with other computing devices. In some examples, the platform 916 also serves to abstract scaling of resources to provide a corresponding level of scale to encountered demand for the resources that are implemented via the platform. Accordingly, in an interconnected device embodiment, implementation of functionality described herein is distributable throughout the system 900. For example, the functionality is implementable in part on the computing device 902 as well as via the platform 916 that abstracts the functionality of the cloud 914.

What is claimed is:

1. A method comprising:
receiving, by a processing device via a network, input data describing interactions of client devices included in a group of client devices;
generating, by the processing device, a first latent vector representation of a first segment of the client devices and a second latent vector representation of a second segment of the client devices using an encoder of a machine learning model;
computing, by the processing device, a change vector based on a difference between the first latent vector representation and the second latent vector representation in a latent space of the machine learning model; and
generating, by the processing device, an indication of an effect of a treatment on a third segment of the client devices based on the change vector using a decoder of the machine learning model.

2. The method as described in claim 1, wherein the machine learning model is trained on the input data without an indication of the treatment.

3. The method as described in claim 1, further comprising regularizing the latent space using a Kullback-Leibler divergence loss.

4. The method as described in claim 1, wherein the machine learning model is a variational autoencoder.

5. The method as described in claim 1, wherein the input data includes categorical data and numerical data.

6. The method as described in claim 5, wherein the machine learning model is trained using a binary cross-entropy loss for the categorical data.

7. The method as described in claim 5, wherein the machine learning model is trained using a mean squared loss for the numerical data.

8. The method as described in claim 5, wherein the machine learning model processes the categorical data using a softmax activation.

9. The method as described in claim 5, wherein the machine learning model processes the numerical data using a linear activation.

10. The method as described in claim 1, wherein at least one of the first segment of the client devices or the second segment of the client devices receives the treatment.

11. A system comprising:
a memory component; and
a processing device coupled to the memory component, the processing device to perform operations comprising:
receiving, via a network, input data describing interactions of client devices included in a group of client devices;
generating a first latent vector representation of a first segment of the client devices and a second latent vector representation of a second segment of the client devices using an encoder of a machine learning model;
computing a change vector based on a difference between the first latent vector representation and the second latent vector representation in a latent space of the machine learning model; and
generating an indication of an effect of a treatment on a third segment of the client devices based on the change vector using a decoder of the machine learning model.

12. The system as described in claim 11, wherein the machine learning model is a variational autoencoder.

13. The system as described in claim 11, wherein the machine learning model is trained using a binary cross-entropy loss for categorical data included in the input data.

14. The system as described in claim 11, wherein the machine learning model is trained using a mean squared loss for numerical data included in the input data.

15. The system as described in claim 11, wherein the machine learning model is trained on the input data without an indication of the treatment.

16. A non-transitory computer-readable storage medium storing executable instructions, which when executed by a processing device, cause the processing device to perform operations comprising:
receiving, via a network, input data describing interactions of client devices included in a group of client devices, the input data includes categorical data and numerical data;
representing the categorical data and the numerical data as a concatenated vector by batch normalizing the categorical data;
generating a first latent vector representation of a first segment of the client devices and a second latent vector representation of a second segment of the client devices based on the concatenated vector using an encoder of a machine learning model; and
generating an indication of an effect of a treatment on a third segment of the client devices based on a difference between the first latent vector representation and the second latent vector representation in a latent space using a decoder of the machine learning model.

17. The non-transitory computer-readable storage medium as described in claim 16, wherein the machine learning model is a variational autoencoder.

18. The non-transitory computer-readable storage medium as described in claim 16, wherein the latent space is regularized using a Kullback-Leibler divergence loss.

19. The non-transitory computer-readable storage medium as described in claim 16, wherein the machine learning model is trained using a binary cross-entropy loss for the categorical data.

20. The non-transitory computer-readable storage medium as described in claim 16, wherein the machine learning model is trained using a mean squared loss for the numerical data.
